(19) United States
(12) Patent Application Publication
(10) Pub. No.: US 2026/0234533 A1
(43) Pub. Date: Aug. 13, 2026

(54) CELL DETACHMENT APPARATUS

(71) Applicants: Canon Kabushiki Kaisha, Tokyo (JP); CANON MEDICAL SYSTEMS CORPORATION, Tochigi (JP)

(72) Inventors: Tomoki HANEISHI, Kita-ku (JP); Naoki NAKAKARUMAI, Taito-ku (JP); Shoji HASHIMOTO, Asao-ku (JP)

(73) Assignees: Canon Kabushiki Kaisha, Tokyo (JP); CANON MEDICAL SYSTEMS CORPORATION, Tochigi (JP)

(21) Appl. No.: 19/438,729

(22) Filed: Jan. 2, 2026

(30) Foreign Application Priority Data
Feb. 7, 2025 (JP) 2025-019390

(51) Int. Cl. C12M 1/26; C12M 3/06

(52) U.S. Cl. CPC C12M 33/12; C12M 27/16

(57) ABSTRACT

A cell detachment apparatus according to an embodiment includes a discharge unit, a collection unit, a tilting mechanism, and a tube. The discharge unit discharges a detachment solution for detaching cells cultured in a culture vessel. The collection unit collects the detached cells in the culture vessel. The tilting mechanism tilts the culture vessel such that the water depth near the collection unit increases. One end of the tube is connected to the discharge unit to supply the detachment solution to the discharge unit. The tube is resistant to pressure required for detaching the cells and includes an expansion/contraction portion that expands and contracts following a change in an orientation of the discharge unit due to tilting of the culture vessel.

### CROSS-REFERENCE TO RELATED APPLICATIONS

This application is based upon and claims the benefit of priority from Japanese Patent Application No. 2025-019390, filed Feb. 7, 2025, the entire contents of which are incorporated herein by reference.

### FIELD

Embodiments described herein relate generally to a cell detachment apparatus.

### BACKGROUND

There are two types of cell culture: open-system culture and closed-system culture. The open-system culture is a common culture method in which, in general, cells are cultured using a dish that is a culture vessel, and an operator opens a lid of the dish to perform operations such as culture medium replacement and subculture. In the open-system culture, since a space in which cells are cultured is basically in an open state, there are safety concerns such as a high risk of bacterial contamination by the operator and a high risk of infection to the operator when viral vectors and the like are used. In the closed-system culture, while cells can be cultured in a closed-system (sealed) culture vessel, operations such as culture medium replacement and cell detachment are performed by the operator in an open state, and thus there is a risk of infection. Therefore, there is a demand for a culture apparatus that allows the culture medium replacement and the cell detachment to be also performed in a closed state. Furthermore, when collecting the cells detached in the culture vessel, it is also required that the operation be performed in a closed state.

### BRIEF DESCRIPTION OF THE DRAWINGS

FIG. 1 is a perspective view of a configuration of a culture apparatus to which a cell detachment apparatus according to an embodiment is applied;

FIG. 2 is a side cross-sectional view of the configuration of the culture apparatus to which the cell detachment apparatus according to the embodiment is applied;

FIG. 3 is a side cross-sectional view of the configuration of the culture apparatus to which the cell detachment apparatus according to the embodiment is applied;

FIG. 4A is a side cross-sectional view of the configuration of the culture apparatus to which the cell detachment apparatus according to the embodiment is applied, and is a diagram illustrating a state in which a supply unit (a cell seeding and culture medium replacement tube) is spaced apart from the liquid surface of a culture medium;

FIG. 4B is a side cross-sectional view of the configuration of the culture apparatus to which the cell detachment apparatus according to the embodiment is applied, and is a diagram in which the supply unit (the cell seeding and culture medium replacement tube) is spaced apart from the liquid surface of the culture medium and a housing is tilted such that a collection unit (a waste liquid collection tube, a cell suspension collection tube) is lower than the supply unit;

FIG. 5A is a side cross-sectional view of the configuration of the culture apparatus to which the cell detachment apparatus according to the embodiment is applied, and is a diagram for describing a tilting mechanism;

FIG. 5B is a side cross-sectional view of the configuration of the culture apparatus to which the cell detachment apparatus according to the embodiment is applied, and is a diagram for describing the tilting mechanism;

FIG. 6A is a side cross-sectional view of the configuration of the culture apparatus to which the cell detachment apparatus according to the embodiment is applied, and is a diagram for explaining a cartridge in which the culture apparatus is housed;

FIG. 6B is a side cross-sectional view of the configuration of the culture apparatus to which the cell detachment apparatus according to the embodiment is applied, and is a diagram for explaining the cartridge in which the culture apparatus is housed;

FIG. 7A is a diagram for describing an expansion/contraction portion of a hard tube located within a culture area in the cell detachment apparatus according to the embodiment;

FIG. 7B is a diagram for describing the expansion/contraction portion of the hard tube located within the culture area in the cell detachment apparatus according to the embodiment;

FIG. 7C is a diagram for describing the expansion/contraction portion of the hard tube located within the culture area in the cell detachment apparatus according to the embodiment;

FIG. 7D is a diagram for describing the expansion/contraction portion of the hard tube located within the culture area in the cell detachment apparatus according to the embodiment;

FIG. 7E is a diagram for describing the expansion/contraction portion of the hard tube located within the culture area in the cell detachment apparatus according to the embodiment;

FIG. 7F is a diagram for describing the expansion/contraction portion of the hard tube located within the culture area in the cell detachment apparatus according to the embodiment; and

FIG. 7G is a diagram for describing the expansion/contraction portion of the hard tube located within the culture area in the cell detachment apparatus according to the embodiment.

### DETAILED DESCRIPTION

A cell detachment apparatus according to an embodiment includes a discharge unit, a collection unit, a tilting mechanism, and a tube. The discharge unit discharges a detachment solution for detaching cells cultured in a culture vessel. The collection unit collects the detached cells in the culture vessel. The tilting mechanism tilts the culture vessel such that the water depth near the collection unit increases. One end of the tube is connected to the discharge unit to supply the detachment solution to the discharge unit. The tube is resistant to pressure required for detaching the cells and includes an expansion/contraction portion that expands and contracts following a change in an orientation of the discharge unit due to tilting of the culture vessel.

Various embodiments will be described hereinafter with reference to the accompanying drawings.

The embodiments are not limited to the following embodiments. The contents described in one embodiment are similarly applied to other embodiments in principle.

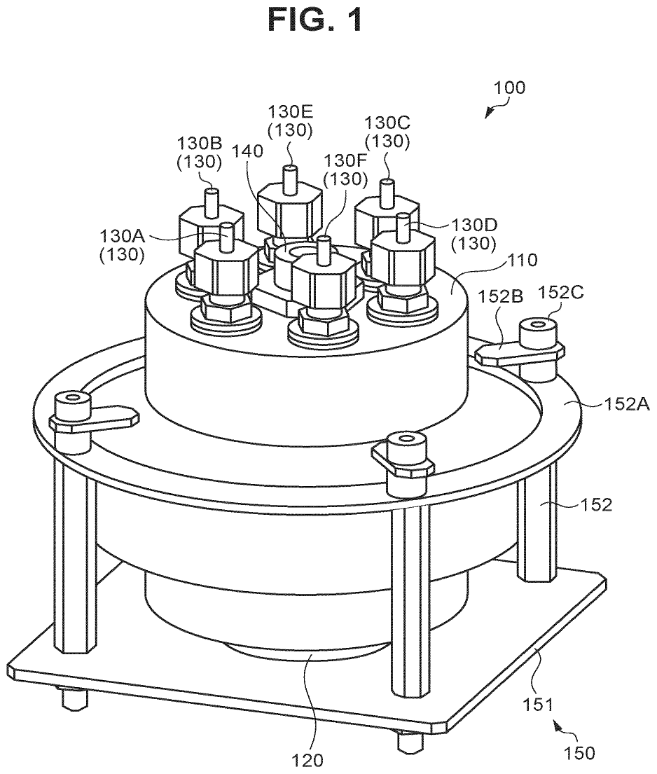

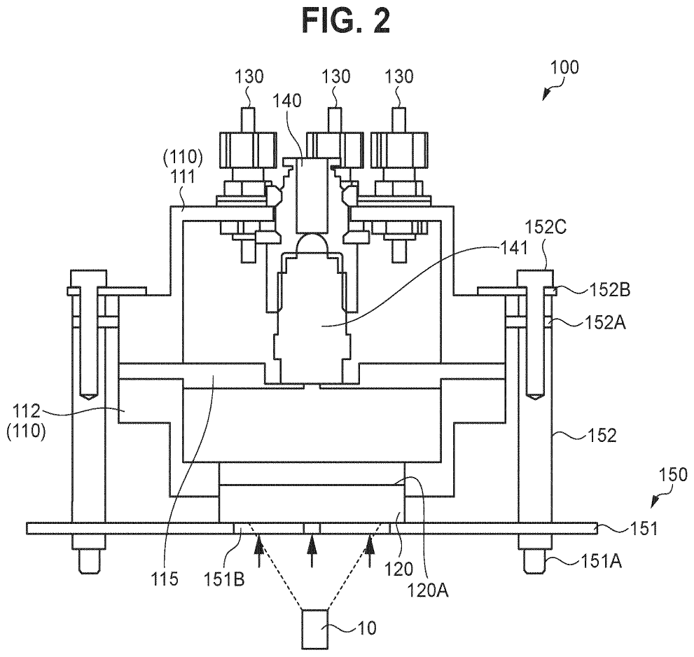

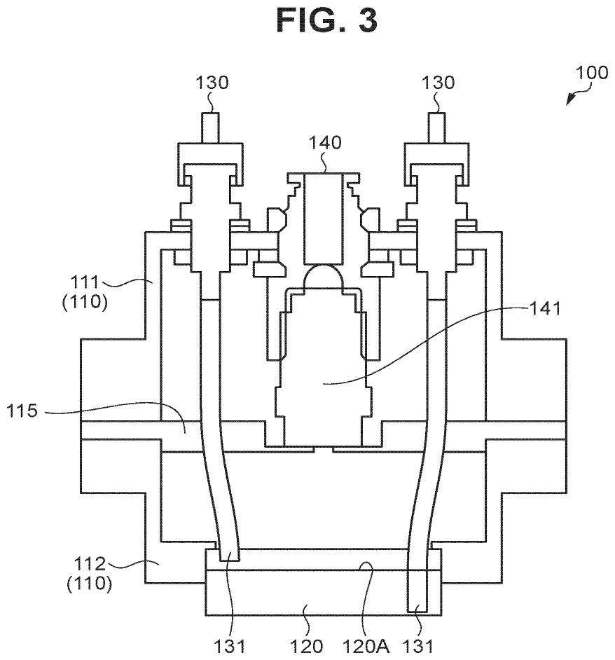

The cell detachment apparatus according to the present embodiment is applied to a culture apparatus 100 illustrated in FIGS. 1 to 3. FIG. 1 is a perspective view of a configuration of the culture apparatus 100 to which the cell detachment apparatus according to the present embodiment is applied. FIGS. 2 and 3 are side cross-sectional views of the configuration of the culture apparatus 100 to which the cell detachment apparatus according to the present embodiment is applied. In FIG. 2, tubes 131 illustrated in FIG. 3 are not illustrated. In FIG. 3, a support member 150 illustrated in FIGS. 1 and 2 is not illustrated.

As illustrated in FIG. 1, the culture apparatus 100 includes a housing 110, a light-shielding plate 115, and a plurality of joints 130.

The housing 110 has a cylindrical part where tubes and the like described below are provided, and includes a first housing part 111 and a second housing part 112 arranged below the first housing part 111. A light-shielding plate 115 is provided between the first housing part 111 and the second housing part 112. In the culture apparatus 100 according to the present embodiment, arbitrary sealing mechanisms are provided between the first housing part 111 and the light-shielding plate 115 and between the light-shielding plate 115 and the second housing part 112, thereby ensuring airtightness of the housing 110.

The first housing part 111, the light-shielding plate 115, and the second housing part 112 have screw holes in the vertical direction and are fastened with screws, for example. In other words, the first housing part 111 and the second housing part 112 support the light-shielding plate 115.

In the culture apparatus 100, to reduce the risk of infection to the operator, tubes for performing culture medium replacement and cell collection in a closed state are used.

The plurality of joints 130 is provided on top of the first housing part 111. As illustrated in FIG. 3, the culture apparatus 100 further includes a plurality of tubes 131. The plurality of tubes 131 is provided inside the housing 110 via the plurality of joints 130.

For example, the plurality of tubes 131 includes a cell seeding and culture medium replacement tube, a waste liquid collection tube, a phosphate-buffered saline (PBS) addition tube, an ethylenediaminetetraacetic acid (EDTA) addition tube, a cell suspension collection tube, and a spare tube. In the example illustrated in FIG. 1, the plurality of joints 130 includes a joint 130A for the cell seeding and culture medium replacement tube, a joint 130B for the waste liquid collection tube, a joint 130C for the PBS addition tube, a joint 130D for the EDTA addition tube, a joint 130E for the cell suspension collection tube, and a joint 130F for the spare tube.

As illustrated in FIG. 3, the plurality of tubes 131 is provided inside the housing 110. As illustrated in FIG. 3, the light-shielding plate 115 has openings for providing the plurality of tubes 131.

As illustrated in FIGS. 2 and 3, a culture vessel 120, which is a dish for culturing cells, is provided at the bottom of the second housing part 112. The culture vessel 120 has a circular shape. The housing 110 is provided on top of the culture vessel 120 in a manner similar to a lid. Distal ends of the plurality of tubes 131 are provided inside the culture vessel 120.

In the culture apparatus 100, a nozzle is used to perform cell detachment in a closed state.

As illustrated in FIGS. 1 to 3, the culture apparatus 100 further includes a joint 140. As illustrated in FIGS. 2 and 3, the culture apparatus 100 further includes a detachment nozzle 141.

The joint 140 is provided in a central portion of an upper part of the first housing part 111. The detachment nozzle 141 is provided inside the housing 110 and connected to the joint 140 at the upper part of the first housing part 111. An opening is provided in the central portion of the light-shielding plate 115, and the distal end of the detachment nozzle 141 is arranged in the opening in the central portion of the light-shielding plate 115.

As illustrated in FIG. 1, the culture apparatus 100 and the culture vessel 120 are supported by the support member 150.

The support member 150 includes a first support member 151 and a second support member 152. The first support member 151 is a member that supports the culture vessel 120 provided at the bottom of the second housing part 112, and the culture vessel 120 is arranged in the central portion of the first support member 151.

For example, the second support member 152 is columnar in shape, and the first support member 151 has a through-hole through which the second support member 152 passes. The second support member 152 is also provided with a member 152A for supporting the side surface of the housing 110, and the outer diameter of the member 152A corresponds to the inner diameter of the housing 110. A member 152B for holding down the first housing part 111 from above is provided at the upper end of the second support member 152. A screw 152C is inserted from the upper end of the second support member 152, and a nut 151A is placed on the bottom surface of the first support member 151 where the through-hole is provided so that the first support member 151 and the second support member 152 are screwed together, thus the culture apparatus 100 and the culture vessel 120 are supported.

A role of the light-shielding plate 115 will be described.

For example, an operator uses a camera to observe the inside of the culture vessel 120. As illustrated in FIG. 2, an observation window 151B is provided in the central portion of the first support member 151, allowing the operator to observe the inside of the culture vessel 120 with a camera 10. The light-shielding plate 115, which is provided in the housing 110, is installed for observation with the camera 10 and is used to prevent the detachment nozzle 141, the tubes 131, and the like from being captured in an image when light is applied from the side of the culture vessel 120.

Next, roles of the plurality of tubes 131 and the detachment nozzle 141 will be described.

The culture apparatus 100 does not have a lid since the medium replacement and cell detachment are performed in a closed state, and therefore the detachment nozzle 141 is used. In the culture apparatus 100, phosphate-buffered saline (PBS) in the form of droplets is sprayed from the detachment nozzle 141 to detach cells adhering to the bottom of the culture vessel 120, and the cells are sucked and collected through the cell suspension collection tube 131.

Specifically, in the culture apparatus 100, first, a process of seeding cells in the culture vessel 120 is performed using the cell seeding and culture medium replacement tube 131. Next, in the culture apparatus 100, a process of replacing the medium is performed on the culture vessel 120 using the cell seeding and culture medium replacement tube 131 and the waste liquid collection tube 131. Since the cells in the culture vessel 120 are contaminated with waste products such as the culture medium, in the culture apparatus 100, a process of washing the cells in the culture vessel 120 by adding PBS, which is a washing solution, using the PBS addition tube 131 and collecting the PBS using the waste liquid collection tube 131 is performed. Then, in the culture apparatus 100, ethylenediaminetetraacetic acid (EDTA) as a solution is added to the culture vessel 120 using the EDTA addition tube 131 to weaken adhesion between the cells and between the cells and the culture vessel 120, and the EDTA is collected using the waste liquid collection tube 131. Then, in the culture apparatus 100, by spraying PBS that is in the form of droplets into the culture vessel 120 using the detachment nozzle 141 and allowing the droplets to hit the inside of the culture vessel 120, a process of detaching the cells adhering to the bottom of the culture vessel 120 is performed. Finally, in the culture apparatus 100, a process of sucking and collecting the cells in the culture vessel 120 using the cell suspension collection tube 131 is performed. At this time, a culture medium is added using the culture medium replacement tube 131, and the cells are collected together with the added culture medium using the waste liquid collection tube 131 and the cell suspension collection tube 131. These processes are performed in a closed state.

The detachment nozzle 141 is an example of a “discharge unit” of the cell detachment apparatus according to the present embodiment. The waste liquid collection tube 131 and the cell suspension collection tube 131 are examples of a “collection unit” of the cell detachment apparatus according to the present embodiment. The housing 110 is an example of a “housing” of the cell detachment apparatus according to the present embodiment.

In the culture apparatus 100, since medium replacement and cell detachment are performed in a closed state, the distal ends of the plurality of tubes 131 are provided inside the culture vessel 120. More specifically, the distal ends of the plurality of tubes 131 are immersed in the liquid inside the culture vessel 120. For this reason, when detached cells in the culture vessel 120 are sucked and collected using the cell suspension collection tube 131, the detached cells may adhere to, for example, the distal end of the tube 131 such as the cell seeding and medium replacement tube 131, resulting in a loss of collected cells.

Therefore, in the present embodiment, in a state in which a supply unit (the cell seeding and culture medium replacement tube 131) is spaced apart from the liquid surface of the culture medium, the housing 110 is tilted such that the collection unit (the waste liquid collection tube 131 and the cell suspension collection tube 131) is located lower than the supply unit, thereby allowing the collection unit to collect the culture medium and cells.

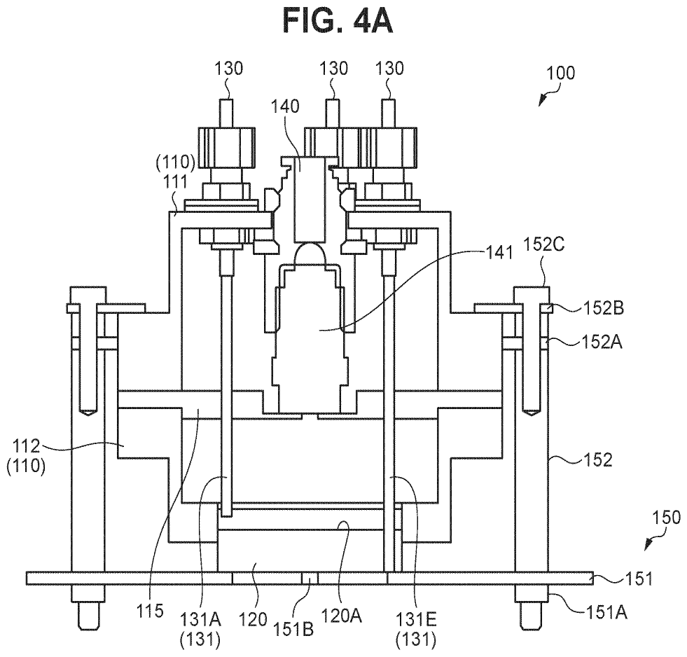

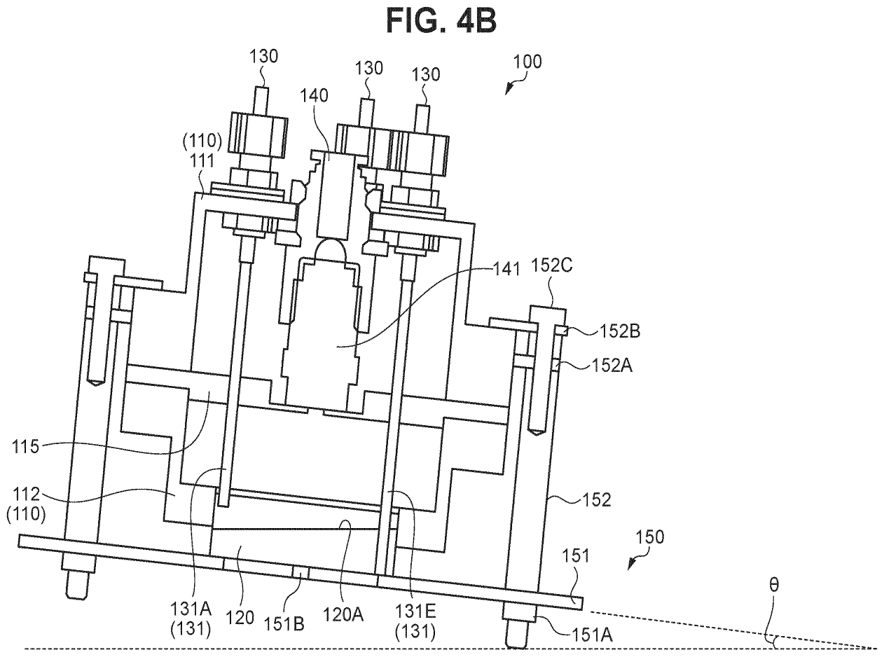

FIG. 4A is a diagram illustrating the state in which the supply unit (the cell seeding and culture medium replacement tube 131) is spaced apart from the liquid surface of the culture medium. FIG. 4B is a diagram illustrating a state in which the housing 110 is tilted such that the collection unit (the waste liquid collection tube 131 and the cell suspension collection tube 131) is lower than the supply unit (the cell seeding and culture medium replacement tube 131) in the state in which the supply unit is spaced apart from the liquid surface of the culture medium. Inside the housing 110, the supply unit (the cell seeding and culture medium replacement tube 131) and the collection unit (the waste liquid collection tube 131 and the cell suspension collection tube 131) are arranged to face each other.

The cell seeding and culture medium replacement tube 131, the waste liquid collection tube 131, the PBS addition tube 131, the EDTA addition tube 131, the cell suspension collection tube 131, and the spare tube 131 are provided inside the housing 110 via the joints 130A to 130F, respectively. In the examples illustrated in FIGS. 4A and 4B, the cell seeding and culture medium replacement tube 131 and the cell suspension collection tube 131 are illustrated as the “cell seeding and culture medium replacement tube 131A” and the “cell suspension collection tube 131E”, respectively.

FIG. 4A is a diagram illustrating a state in which the supply unit (the cell seeding and culture medium replacement tube 131) is spaced apart from a liquid surface 120A of the culture medium. In the example illustrated in FIGS. 3 and 4A, a discharge port of the supply unit (the distal end of the cell seeding and culture medium replacement tube 131) is disposed apart from the liquid surface 120A of the culture medium.

FIG. 4B is a diagram illustrating a state in which the supply unit (the cell seeding and culture medium replacement tube 131) is spaced apart from a liquid surface 120A of the culture medium and in which the housing 110 is tilted such that the collection unit (the waste liquid collection tube 131 and the cell suspension collection tube 131) becomes lower than the supply unit. For example, an angle θ at which the housing 110 is tilted with respect to the horizontal plane is one degree or more and 60 degrees or less. In the present embodiment, the housing 110 is tilted such that a suction port of the collection unit (the distal end of the waste liquid collection tube 131 and the distal end of the cell suspension collection tube 131) is lower than the discharge port of the supply unit. At this time, a cell suspension moves toward the collection unit. In the present embodiment, in a state where the housing 110 is tilted at the angle θ with respect to the horizontal plane, the culture medium and cells are collected by the collection unit. Accordingly, in the present embodiment, in the culture of cells in a closed state, since the cell suspension is moved toward the collection unit before the culture medium and cells are collected, the efficiency of cell collection is increased.

In the examples illustrated in FIGS. 4A and 4B, the discharge port of the supply unit is spaced apart from the liquid surface 120A of the culture medium even before the housing 110 is tilted. However, this is not limiting. For example, the discharge port of the supply unit may be spaced apart from the liquid surface 120A of the culture medium after the housing 110 is tilted.

In the present embodiment, in order to further increase the efficiency of cell collection, a culture medium may be added again, and the added culture medium may be collected. For example, after the culture medium and cells are collected by the collection unit (the waste liquid collection tube 131 and the cell suspension collection tube 131), the culture medium is added to the culture vessel 120 by the supply unit (the cell seeding and culture medium replacement tube 131). Then, the added culture medium is collected by the collection unit (in this case, the waste liquid collection tube 131).

Next, a mechanism for tilting the housing 110 of the culture apparatus 100 will be described.

The culture apparatus 100 further includes a tilting mechanism. The tilting mechanism tilts the housing 110 of the culture apparatus 100 such that the collection unit (the waste liquid collection tube 131 and the cell suspension collection tube 131) is lower than the supply unit (the cell seeding and medium replacement tube 131) in a state in which the supply unit is spaced apart from the liquid surface 120A of the culture medium. In other words, the tilting mechanism tilts the culture vessel 120 such that the water depth near the collection unit increases.

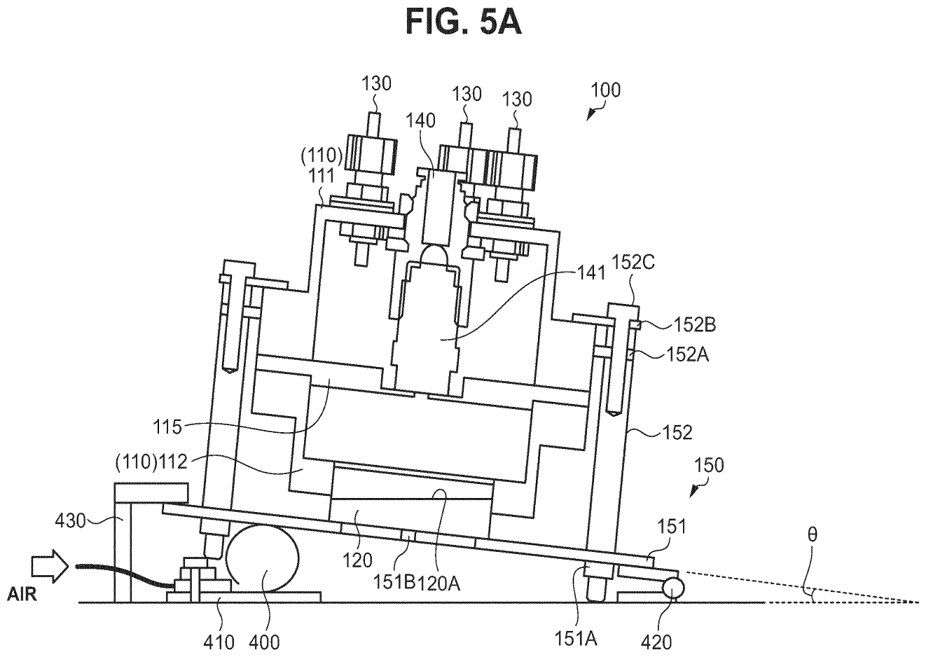

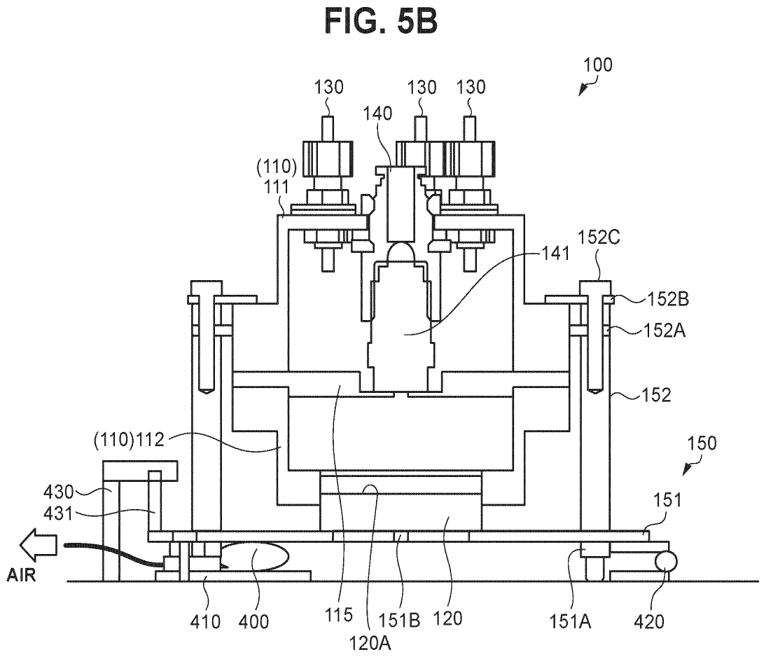

FIGS. 5A and 5B are diagrams for describing a configuration of the tilting mechanism. In FIGS. 5A and 5B, the tubes 131 illustrated in FIG. 4 are omitted.

FIG. 5A is a diagram illustrating processing of tilting the culture apparatus 100 performed by the tilting mechanism. In the example illustrated in FIG. 5A, the tilting mechanism includes a balloon member 400, support members 410 and 420, and a stopper member 430. The balloon member 400 is arranged on the underside of the culture apparatus 100. The support member 410 is a member with a path for introducing and evacuating air, and supports the balloon member 400. When air flows into the balloon member 400 via the support member 410, the balloon member 400 arranged on the underside of the culture apparatus 100 is inflated. The inflated balloon member 400 tilts the housing 110 of the culture apparatus 100 at the angle θ with respect to the horizontal plane. The support member 420 is a member with a hinge, and supports the housing 110 that tilts about the hinge serving as a fulcrum. The stopper member 430 is a member for stopping the tilting of the housing 110.

As described above, in the present embodiment, the tilting mechanism supplies air to the balloon member 400 on the underside of the housing 110 of the culture apparatus 100, thereby tilting the housing 110. The balloon member 400 is not particularly limited, and may be substituted by an air cylinder, for example.

FIG. 5B is a diagram illustrating a state in which the processing performed by the tilting mechanism is released. In the example illustrated in FIG. 5B, the tilting mechanism further includes a coil spring 431. The coil spring 431 is connected to the stopper member 430 and the first support member 151. Evacuating the air from inside the balloon member 400 reduces a lifting force, and a force of the coil spring 431 supports the housing 110 of the culture apparatus 100 in a horizontal state. Alternatively, when evacuating the air from inside the balloon member 400 via the support member 410, the coil spring 431 may push the first support member 151, thereby contracting the balloon member 400 and supporting the housing 110 in a horizontal state.

The tilting mechanism (the balloon member 400, the support members 410 and 420, and the stopper member 430) is an example of the “tilting mechanism” of the cell detachment apparatus according to the present embodiment.

The culture apparatus 100 includes a tube with one end thereof connected to the detachment nozzle 141 via the joint 140, and a detachment solution (for example, the above-described PBS in the form of droplets) for detaching cells cultured in the culture vessel 120 is supplied to the detachment nozzle 141 via the tube. In this case, the detachment solution to which a pressure required for cell detachment is applied needs to be supplied to the detachment nozzle 141. Thus, as the tube connected to the detachment nozzle 141, a hard tube that is made of a material that can maintain its shape against the pressure required for cell detachment is used. For example, the hard tube is made of a material harder than a silicone tube. Such a hard tube that meets pressure-resistant requirements for detachment spraying and enables tilting of the culture vessel 120 when being connected to the detachment nozzle 141 is required.

Therefore, the cell detachment apparatus according to the present embodiment includes the detachment nozzle 141 that is a discharge unit, the collection unit (the waste liquid collection tube 131 and the cell suspension collection tube 131), the tilting mechanism (the balloon member 400, the support members 410 and 420, and the stopper member 430), and the hard tube to enable tilting of the culture vessel 120 in cell culture in a closed state and to increase the efficiency of cell collection. The discharge unit discharges the detachment solution in the form of mist to detach the cells cultured in the culture vessel 120. The collection unit collects the detached cells in the culture vessel 120. As described above, the tilting mechanism tilts the culture vessel 120 such that the water depth near the collection unit increases. One end of the hard tube is connected to the discharge unit to supply the detachment solution to the discharge unit. The hard tube is resistant to the pressure required for cell detachment and includes an expansion/contraction portion that expands and contracts following a change in the orientation of the discharge unit due to tilting of the culture vessel 120.

Accordingly, the cell detachment apparatus according to the present embodiment enables the tilting of the culture vessel 120 in the culture of cells in a closed state, and tilts the culture vessel 120 such that the collection unit becomes lower than the supply unit in the state in which the supply unit is spaced apart from the liquid surface of the culture medium, thereby collecting the culture medium and cells by the collection unit.

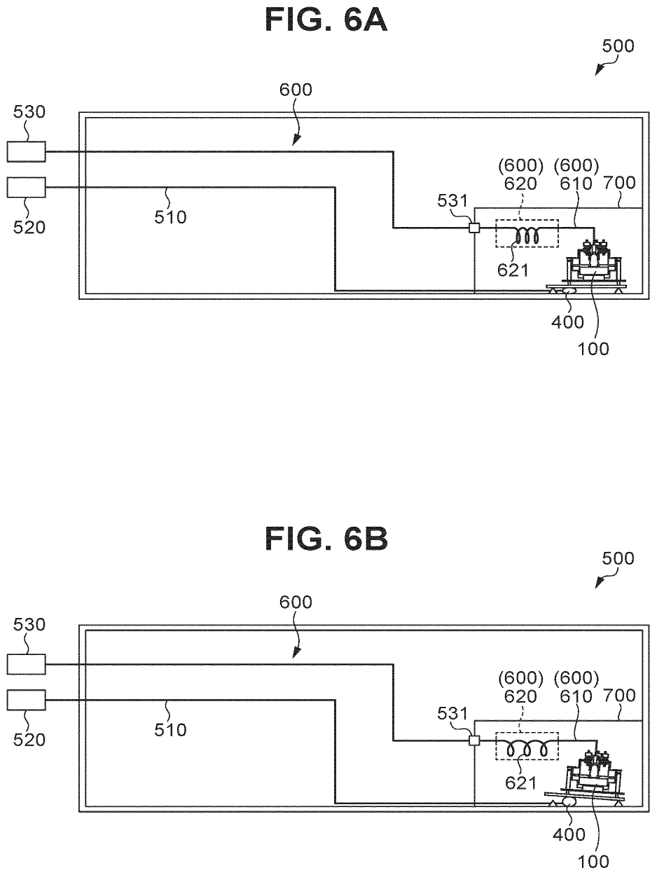

FIGS. 6A and 6B are diagrams for describing a cartridge 500 in which the culture apparatus 100 is housed.

As illustrated in FIG. 6A, with regard to the culture apparatus 100, the cartridge 500 has a flow path for supplying the culture medium to the supply unit (the cell seeding and culture medium replacement tube 131), a flow path for supplying droplets to the discharge unit (the detachment nozzle 141), a flow path for collecting cells by the collection unit (the waste liquid collection tube 131 and the cell suspension collection tube 131), and a flow path 510 for supplying air to the balloon member 400 of the tilting mechanism.

The cartridge 500 houses, as the culture apparatus 100, a culture apparatus for expansion culture and a culture apparatus for main culture. More specifically, the cartridge 500 includes a culture area for housing the culture apparatus for the expansion culture as the culture apparatus 100, and a culture area for housing the culture apparatus for the main culture as the culture apparatus 100.

For example, culture is performed twice, and the expansion culture and the main culture are performed at different culture stages within the cartridge. Taking iPS cell culture as an example, culturing of cells before they become iPS cells is performed as the expansion culture. After the expansion culture, genes are introduced to generate iPS cells, and as the main culture, culture for increasing cells that have become iPS cells is performed. In the example illustrated in FIGS. 6A and 6B, as the culture apparatus 100, the culture apparatus 100 for the main culture is illustrated, and a culture area 700 where the culture apparatus 100 for the main culture is housed is illustrated. The culture apparatus for the expansion culture and the culture area where the culture apparatus for the expansion culture is housed are not illustrated.

In this case, the cartridge 500 includes a flow path 510 for supplying air to the balloon member 400 of the tilting mechanism. The air is supplied, for example, by a pump 520 provided outside the cartridge 500. In FIG. 6A, because the balloon member 400 arranged on the underside of the culture apparatus 100 is contracted, the culture apparatus 100 maintains a horizontal state in the cartridge 500.

The cartridge 500 also includes a hard tube 600 with one end thereof connected to the detachment nozzle 141 via the joint 140 to supply the detachment solution to the detachment nozzle 141. In the example illustrated in FIGS. 6A and 6B, the hard tube 600 connected to the detachment nozzle 141 of the culture apparatus 100 for the main culture is illustrated, whereas the hard tube 600 connected to the detachment nozzle 141 of the culture apparatus 100 for the expansion culture is not illustrated.

The hard tube 600 is an example of the “tube” of the cell detachment apparatus according to the present embodiment.

The hard tube 600 is resistant to the pressure required for cell detachment. For example, the hard tube 600 is resistant to the pressure applied to the detachment solution discharged from the detachment nozzle 141.

For example, the hard tube 600 is made of a material that can maintain its shape against the pressure required for cell detachment. For example, the material of the hard tube 600 is harder than a silicone tube. Specifically, the material of the hard tube 600 may be ethylene tetrafluoroethylene (ETFE), which is a fluororesin obtained by copolymerizing tetrafluoroethylene and ethylene. Alternatively, the material of the hard tube 600 may be perfluoroalkoxy (PFA), which is a fluororesin obtained by copolymerizing tetrafluoroethylene and perfluoroalkoxyethylene. Alternatively, the material of the hard tube 600 may be polytetrafluoroethylene (PTFE), which is a crystalline resin made of fluorine and carbon.

The hard tube 600 includes a part provided between a cylinder 530 outside the cartridge 500 and a joint 531 installed on a partition wall of the culture area 700, and a part provided between the joint 531 in the culture area 700 and the detachment nozzle 141 in the culture area 700. The detachment solution is supplied to the hard tube 600, for example, by the cylinder 530 outside the cartridge 500. The hard tube 600 supplies the detachment solution to which the pressure required for cell detachment is applied to the detachment nozzle 141 in the culture area 700. Specifically, the hard tube 600 supplies the detachment solution to the detachment nozzle 141 while maintaining the pressure applied to the detachment solution by the cylinder 530 that supplies the detachment solution.

The cylinder 530 is an example of a “detachment solution supply source” of the cell detachment apparatus according to the present embodiment.

The part of the hard tube 600 provided in the culture area 700 includes an expansion/contraction portion 620 that expands and contracts following a change in the orientation of the detachment nozzle 141 due to tilting of the culture vessel 120. Specifically, the expansion/contraction portion 620 is provided in the hard tube 600 within the culture area 700 where the culture vessel 120 is installed. For example, the hard tube 600 has a main portion 610 extending in a first direction (the longitudinal direction of the cartridge 500 in FIG. 6A), and the expansion/contraction portion 620 having a curved portion 621 that bends at an angle of less than 90 degrees in a direction intersecting the first direction. In other words, the curved portion 621 can bend gently at an angle smaller than a right angle with respect to the linear main portion 610 extending in the first direction. If the angle is too large, gas such as air may accumulate in the hard tube 600, which may result in insufficient spray performance of the detachment nozzle 141. The shape and type of the curved portion 621 will be described below.

FIG. 6B is a diagram illustrating a state in which the housing 110 of the culture apparatus 100 is tilted in the cartridge 500. When air flows into the balloon member 400 via the flow path 510, the balloon member 400 of the tilting mechanism arranged on the underside of the culture apparatus 100 becomes inflated, and the inflated balloon member 400 tilts the housing 110 of the culture apparatus 100. In other words, in the culture apparatus 100, when the tilting mechanism tilts the housing 110, the housing 110 is tilted within the cartridge 500.

In this case, when the culture vessel 120 is tilted, the detachment nozzle 141 is tilted in accordance with the tilting of the culture vessel 120, and the hard tube 600 is pulled in accordance with the change in the orientation of the detachment nozzle 141. Thus, the expansion/contraction portion 620 of the hard tube 600 expands in accordance with the change in the orientation of the detachment nozzle 141.

At this time, the cylinder 530 outside the cartridge 500 supplies the detachment solution to the hard tube 600, and the hard tube 600 supplies the detachment solution to the detachment nozzle 141 while maintaining the pressure applied to the detachment solution by the cylinder 530. Subsequently, in a state where the culture apparatus 100 is tilted, the culture medium and cells are collected by the collection unit (the waste liquid collection tube 131 and the cell suspension collection tube 131).

On the other hand, as illustrated in FIG. 6A, in a state in which the processing performed by the tilting mechanism is released, the housing 110 is supported in a horizontal state, so that the hard tube 600 is pushed in, and the expansion/contraction portion 620 of the hard tube 600 contracts following a change in the orientation of the detachment nozzle 141.

In the present embodiment, the material of the hard tube 600 may be ETFE, PFA, PTFE, or the like, but is not limited to these. For example, the hard tube 600 may have hardness that prevents expansion when subjected to the pressure required for cell detachment. For example, the Shore D hardness of the hard tube 600 can be 50 to 80 [HS].

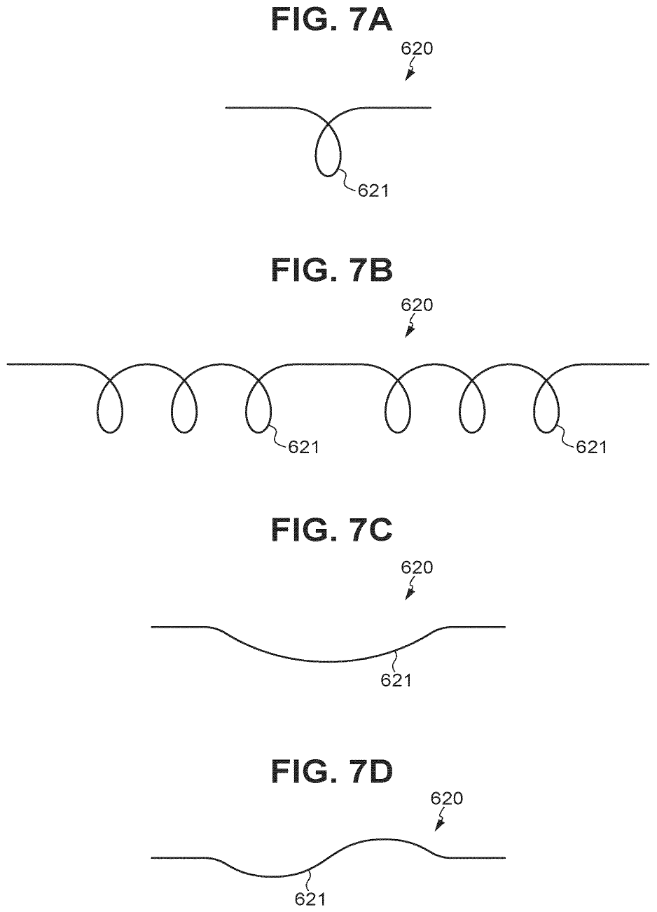

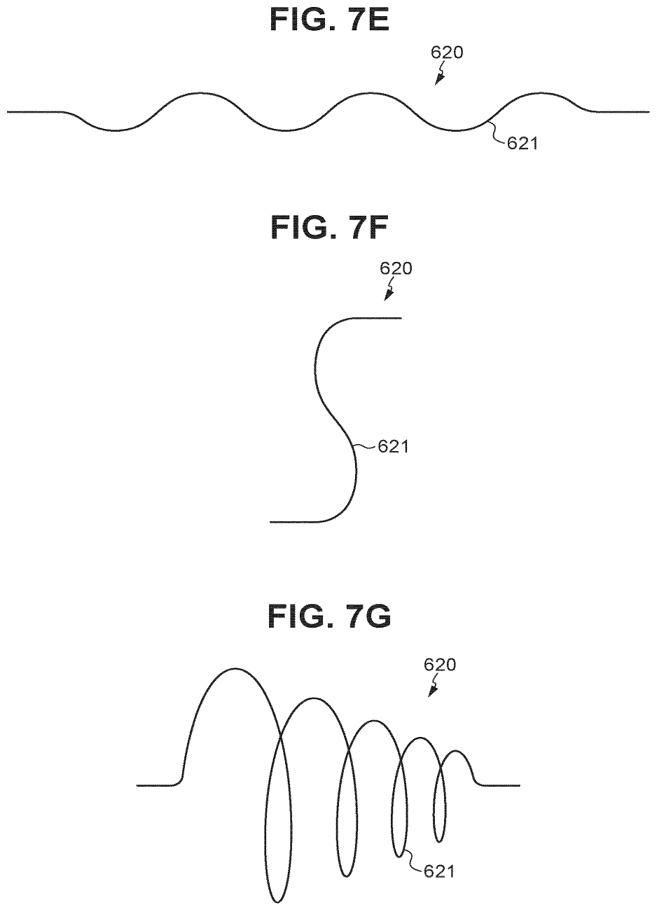

FIGS. 7A to 7G are diagrams for describing the expansion/contraction portion 620 of the hard tube 600 within the culture area 700 in the cell detachment apparatus according to the present embodiment.

In the example illustrated in FIG. 7A, the expansion/contraction portion 620 of the hard tube 600 includes one curved portion 621 within the culture area 700. In FIG. 7A, for example, the curved portion 621 has a coil shape with one turn.

In the example illustrated in FIG. 7B, the expansion/contraction portion 620 of the hard tube 600 includes a plurality of curved portions 621 within the culture area 700. In FIG. 7B, for example, the expansion/contraction portion 620 includes a plurality of curved portions 621 formed in a spiral shape. Specifically, a spiral tube includes three coil-shaped curved portions 621 coupled in a spiral manner as a set, and two such spiral tubes are provided spaced apart. The hard tube 600 illustrated in each of FIGS. 6A and 6B includes one spiral tube in which three coil-shaped curved portions 621 are coupled in a spiral manner.

In the example illustrated in FIG. 7C, the expansion/contraction portion 620 of the hard tube 600 includes one curved portion 621 within the culture area 700. In FIG. 7C, for example, the curved portion 621 is formed in an R-shape (arc shape).

In the example illustrated in FIG. 7D, the expansion/contraction portion 620 of the hard tube 600 has a plurality of curved portions 621 within the culture area 700. In FIG. 7D, for example, the plurality of curved portions 621 is formed in an S-shape by two curved portions 621.

In the example illustrated in FIG. 7E, the expansion/contraction portion 620 of the hard tube 600 includes a plurality of curved portions 621 within the culture area 700. In FIG. 7E, for example, the plurality of curved portions 621 is formed in a wave shape by six curved portions 621.

In the example illustrated in FIG. 7F, the expansion/contraction portion 620 of the hard tube 600 includes a plurality of curved portions 621 within the culture area 700. The plurality of curved portions 621 is formed in an S-shape by two curved portions 621. In the example illustrated in FIG. 7D, the S-shape is formed in the first direction, whereas in the example illustrated in FIG. 7F, the S-shape is formed in a second direction perpendicular to the first direction.

In the example illustrated in FIG. 7G, the expansion/contraction portion 620 of the hard tube 600 includes a plurality of curved portions 621 within the culture area 700. In FIG. 7G, for example, the expansion/contraction portion 620 includes the plurality of curved portions 621 formed in a spiral shape. Specifically, the expansion/contraction portion 620 illustrated in FIG. 7G includes a spiral tube formed in a shape similar to a spiral shell by coupling four coil-shaped curved portions 621 whose diameters gradually decrease from left to right.

As described above, the cell detachment apparatus according to the present embodiment includes the detachment nozzle 141 that is a discharge unit, the collection unit (the waste liquid collection tube 131 and the cell suspension collection tube 131), the tilting mechanism (the balloon member 400, the support members 410 and 420, and the stopper member 430), and the hard tube. The discharge unit discharges the detachment solution in the form of mist to detach the cells cultured in the culture vessel 120. The collection unit collects the detached cells in the culture vessel 120. The tilting mechanism tilts the culture vessel 120 such that the water depth near the collection unit increases. One end of the hard tube is connected to the discharge unit to supply the detachment solution to the discharge unit. The hard tube is resistant to the pressure required for cell detachment and includes the expansion/contraction portion that expands and contracts following a change in the orientation of the discharge unit due to the tilting of the culture vessel 120. Accordingly, the cell detachment apparatus according to the present embodiment allows the tilting of the culture vessel 120 in culture of cells in a closed state, and increases the efficiency of cell collection.

In the above-described embodiment, the case has been described in which the discharge port of the supply unit (the distal end of the cell seeding and culture medium replacement tube 131) is spaced apart from the liquid surface 120A of the culture medium when the culture apparatus 100 is in a horizontal state (before being tilted). However, in the above-described embodiment, the discharge port of the supply unit (the distal end of the cell seeding and culture medium replacement tube 131) may be located within the culture medium when the culture apparatus 100 is in the horizontal state (before being tilted), and the discharge port of the supply unit may be spaced apart from the liquid surface 120A when the culture apparatus 100 is tilted.

In the above-described embodiment, when the culture vessel 120 is tilted, the detachment nozzle 141 is tilted in accordance with the tilting of the culture vessel 120, and the hard tube 600 is pulled in accordance with the change in the orientation of the detachment nozzle 141. Thus, the expansion/contraction portion 620 of the hard tube 600 expands in accordance with the change in the orientation of the detachment nozzle 141. However, the present embodiment is not limited to this.

For example, when the culture vessel 120 is tilted, the detachment nozzle 141 is tilted in accordance with the tilting of the culture vessel 120, and the hard tube 600 may be pushed in accordance with the change in the orientation of the detachment nozzle 141. Thus, the expansion/contraction portion 620 of the hard tube 600 contracts in accordance with the change in the orientation of the detachment nozzle 141. In this case, in a state in which the processing performed by the tilting mechanism is released, the housing 110 is supported in the horizontal state, so that the hard tube 600 is pulled, and the expansion/contraction portion 620 of the hard tube 600 expands following a change in the orientation of the detachment nozzle 141.

According to at least one of the embodiments described above, it is possible to increase the efficiency of cell collection in culture of cells in a closed state.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A cell detachment apparatus comprising:
a discharge unit configured to discharge a detachment solution for detaching cells cultured in a culture vessel;
a collection unit configured to collect the detached cells in the culture vessel;
a tilting mechanism configured to tilt the culture vessel such that a water depth near the collection unit increases; and
a tube having one end connected to the discharge unit, the tube being configured to supply the detachment solution to the discharge unit,
wherein the tube is resistant to pressure required for detaching the cells and includes an expansion/contraction portion that expands and contracts following a change in an orientation of the discharge unit due to tilting of the culture vessel.

2. The cell detachment apparatus according to claim 1, further comprising a housing in which the culture vessel, the discharge unit, and the collection unit are provided,
wherein the tilting mechanism tilts the housing.

3. The cell detachment apparatus according to claim 1,
wherein the tube has a main portion extending in a first direction, and
wherein the expansion/contraction portion has a curved portion that bends in a direction intersecting the first direction.

4. The cell detachment apparatus according to claim 3, wherein the expansion/contraction portion has one or more curved portions.

5. The cell detachment apparatus according to claim 4, wherein the expansion/contraction portion includes a plurality of curved portions formed in a spiral shape.

6. The cell detachment apparatus according to claim 1, wherein the expansion/contraction portion is provided in the tube located within a culture area where the culture vessel is installed.

7. The cell detachment apparatus according to claim 1, wherein the tube supplies the detachment solution to which the pressure required for detaching the cells is applied to the discharge unit.

8. The cell detachment apparatus according to claim 1, wherein the tube supplies the detachment solution to the discharge unit while maintaining pressure applied to the detachment solution by a detachment solution supply source configured to supply the detachment solution.

9. The cell detachment apparatus according to claim 1, wherein the tube is made of a material capable of maintaining a shape of the tube against the pressure required for detaching the cells.

10. The cell detachment apparatus according to claim 9, wherein the material of the tube is harder than a silicone tube.

11. The cell detachment apparatus according to claim 1, wherein the tube has hardness that prevents expansion when subjected to the pressure required for detaching the cells.

12. The cell detachment apparatus according to claim 10, wherein the material of the tube is ethylene tetrafluoroethylene (ETFE), perfluoroalkoxy (PFA), or polytetrafluoroethylene (PTFE).

13. The cell detachment apparatus according to claim 11, wherein a Shore D hardness of the tube is 50 to 80 [HS].
